(19) United States
(12) Patent Application Publication
(10) Pub. No.: US 2004/0092838 A1
(43) Pub. Date: May 13, 2004

(54) Signal transmitting belt

(75) Inventor: Chun-Mu Huang, Taipei (TW)

Correspondence Address: Haverstock & Owens, LLP., 162 North Wolfe Road, Sunnyvale, CA, 94086, US

(73) Assignee: Echowell Electronic Co., Ltd.

(21) Appl. No.: 10/698,181

(22) Filed: Oct. 30, 2003

(30) Foreign Application Priority Data
Oct. 30, 2002 (TW) 091132211

(51) Int. Cl. A61B 5/0402

(52) U.S. Cl. 600/519

(57) ABSTRACT

A signal transmitting belt for transmitting a heartbeat signal to a heart rate monitor is provided, which shows a cardiorespiratory information. The signal transmitting belt includes: a main body comprising a signal transmitter for transmitting the heartbeat signal, an insulating chest belt, a connecting element connecting the main body and the insulating chest belt for sustaining a tension therebetween, and a conductive chest belt mounted on the insulating chest belt for contacting a chest of a user so as to detect the heartbeat signal and then transmit the heartbeat signal to the signal transmitter.

### FIELD OF THE INVENTION

The present invention is related to a signal transmitting belt, and more particularly, to a signal transmitting belt for transmitting a heartbeat signal to a heart rate monitor so as to show cardiorespiratory information.

### BACKGROUND OF THE INVENTION

A heart rate monitor is very useful for athletes or those people who want to maintain a healthy level of exercise. A heart rate monitor tells the user whether his heart rate is in the proper range for the type of activity he is doing. The software inside will guide the user through a complete exercise cycle and keeps the user on track for his workout based on the personal data such as gender, age, weight, and height. During the workout), the monitor measure and displays the user's heart rate and also shows the number of calories consumed and the percentage of fat burned. Other features of heart rate monitor include an alarm clock, a stopwatch, and the memory function for recalling the user's heart rate history. The heart rate monitor is usually worn as a wristwatch with an additional heart rate chest belt to transmit data to the receiver watch.

Generally, there are two types of heart rate chest belt, the single type and the combined type. The single type of heart rate chest belt is formed integrally including the belt and the transmitter. The combined type of heart rate chest belt is combined by three pieces, the main body having a transmitter inside, the insulating chest belt, and the conductive chest belts. The heart rate chest belt detects the heart rate and transmits the heartbeat signal to the receiver watch so that the user is able to be aware of his own cardiorespiratory function while he is exercising or doing certain activities. Please refer to FIG. 1. FIG. 1 is a diagram illustrating the conventional structure of the heart rate chest belt according to the prior art. The signal transmitting belt 10 for transmitting a heartbeat signal to a heart rate monitor includes the main body 11, the engaging slot 12, the conductive chest belt 13, the engaging element 14, the insulating chest belt 15, and the buckle element 16. The engaging slot 12 is mounted at two sides of the main body 11, while the engaging element 14 is mounted at one side of the conductive chest belt 13. Through the engagement between the engaging slot 12 and the engaging element 14, the main body 11 is connected with the conductive chest belt 13. The mail body 11 aid the conductive chest belt 13 are usually combined with the insulating chest belt 15 to complete a heart rate chest belt. The signal transmitting belt 10 can be worn and fixed on the user's chest by buckling the buckle element 16 on the insulating chest belt 15 so that the user's heart rate can be detected.

However, the conductive chest belt 13 is usually made of plastic or rubber material containing conductive substances, which seriously decreases the stretchable property of the conductive chest belt 13. Yet since the conductive chest belt 13 is combined with the insulating chest belt 15, it often has to sustain the strong pulling strength between the main body 11 and the insulating chest belt 15 when the user wears the signal transmitting belt 10. Therefore, the engaging element 14 is often damaged owing to the pulling strength. When this happens, not only the outside housing of the main body 11 but also the conductive chest bolt 13 has to be replaced, which costs too high and is very inconvenient for the user. In addition, the engagement between engaging slot 12 and the engaging element 14 results in the low flexibility of the signal transmitting belt 10 since the engagement point hinders the belt from freely rotating. Thus, the user can not feel completely comfortable when wearing the signal transmitting belt it).

According to the above, it is clear that how to enhance the structure stability and flexibility of the signal transmitting belt has become a major problem waited to be solved in the industry. In order to overcome the drawbacks in the prior art, a signal transmitting belt for a heart rate monitor, which has a more stable structure and a longer life span, is provided.

### SUMMARY OF THE INVENTION

The main purpose of the present invention is to provide a signal transmitting belt for transmitting a heartbeat signal to a heart rate monitor so as to show the cardiorespiratory information of the user. The signal transmitting belt provided in the present invention has a more stable structure to sustain the drawing tension between the main body and the insulating chest belt so that the life span is prolonged.

According to one aspect of the present invention, a signal transmitting bolt for transmitting a heartbeat signal to a heart rate monitor so as to show a cardiorespiratory information, includes: a main body comprising a signal transmitter for transmitting the heartbeat signal, an insulating chest belt, a connecting element connecting the main body and the insulating chest belt for sustaining a tension therebetween, and a conductive chest belt mounted on the insulating chest belt for contacting a chest of a user so as to detect the heartbeat signal and then transmit the heartbeat signal to the signal transmitter.

In accordance with the present invention, the connecting element is a connecting shaft.

Preferably, the connecting element is a round shaft so that the insulating chest belt is rotatable around the main body.

Preferably, the connecting element is a rectangular shaft.

Preferably, the conductive chest belt transmits the heartbeat signal from the user to the signal transmitter through the connecting element.

Preferably, the conductive chest bell further comprises a protrudent portion having a signal transmitting hole thereon for transmitting the heartbeat signal therethrough.

Preferably, the signal transmitting belt further comprises a first spring mounted on the connecting element and a first screw screwed onto the signal transmitting hole.

Preferably, the first spring is screwed onto the conductive chest belt by the first screw for transmitting the heartbeat signal to the connecting element.

Preferably, the signal transmitting belt further includes a washer screwed together with the first spring by the first screw so that the first spring is screwed onto the conductive chest belt under an even pressure.

Preferably, the conductive chest belt further comprises an axle hole for mounting the connecting element therethrough.

Preferably, the first spring is mounted inside the axle hole of the insulating chest belt.

Preferably, the connecting element has a flange thereon for being pushed against the first spring mounted inside the axle hole.

Preferably, the insulating chest belt and the main body are assembled together both through side portions thereof to cooperatively form a passing hole for passing through the connecting element

Preferably, the signal transmitting belt further comprises a second spring mounted on the connecting element and a second screw mounted on the main body.

Preferably, the second spring is looped around the connecting element for transmitting the heartbeat signal from the connecting element to the signal transmitter of the main body.

Preferably, the main body further comprises an upper cover and a lower cover.

Preferably, the upper cover and the lower cover further comprise a fixing bearing and an auxiliary bearing respectively for fixedly mounting the connecting element therebetween.

Preferably, the signal transmitting belt further comprises a skidproof slice mounted in an indentation of the main body for preventing the connecting element from coming off.

Preferably, the insulating chest belt has a rotating angle of 180° corresponding to the main body for providing a flexibility to the signal transmitting belt when being used.

According to another aspect of the present invention, a signal transmitting belt for transmitting a heartbeat signal to a heart rate monitor so as to show a cardiorespiratory information for a user, includes: a main body comprising a signal transmitter therein for transmitting the heartbeat signal, an insulating chest belt having a conductive sensing element mounted therein for contacting a chest of the user so as to detect the heartbeat signal and then transmit the heartbeat signal to the signal transmitter, and a connecting element connecting the main body and the insulating chest belt for sustaining a tension therebetween.

The foregoing and other features and advantages of the present invention will be more clearly understood through the following descriptions with reference to the drawings, wherein:

### BRIEF DESCRIPTION OF THE DRAWINGS

FIG. 1 is a diagram illustrating the conventional structure of the signal transmitting belt according to the prior art:

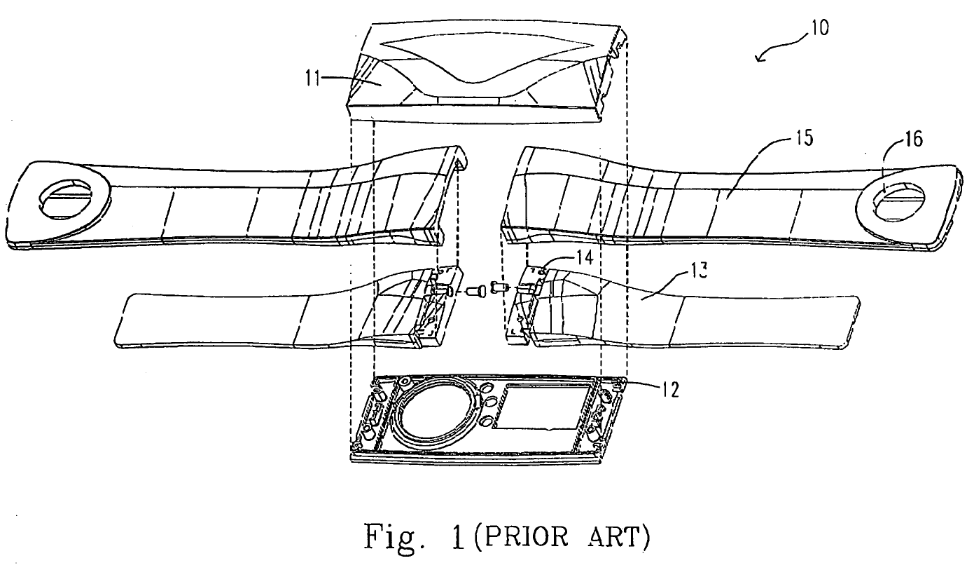

FIG. 2 is a diagram illustrating the heart rate monitor device according to a preferred embodiment of the present invention;

FIG. 3 is a diagram illustrating the structure of the signal transmitting belt according to a preferred embodiment of the present invention;

FIG. 4 is a diagram illustrating the structure of the connecting shaft and the conductive chest belt according to a preferred embodiment of the present invention;

FIG. 5 is a diagram showing the outside appearance of the signal transmitting belt according to a preferred embodiment of the present invention;

FIG. 6 is a diagram illustrating the structure of the signal transmitting belt according to another preferred embodiment of the present invention;

FIG. 7 is a diagram illustrating the structure of the connecting element and the conductive chest belt according to a preferred embodiment of the present invention;

FIG. 8 is diagram illustrating the structure of the first shaft body and the conductive chest belt used in the manufacturing method for a signal transmitting belt according to a preferred embodiment of the present invention;

FIG. 9 is diagram illustrating the combined structure of the first shaft body and the conductive chest belt used in the manufacturing method for a signal transmitting belt according to a preferred embodiment of the present invention,

FIG. 10 is a diagram illustrating the structure of the signal transmitting belt before the manufacturing is accomplished according to a preferred embodiment of the present invention; and

FIG. 11 is a diagram illustrating the structure of the connecting element and the lower cover according to a preferred embodiment of the present invention.

### DETAILED DESCRIPTION OF THE PREFERRED EMBODIMENT

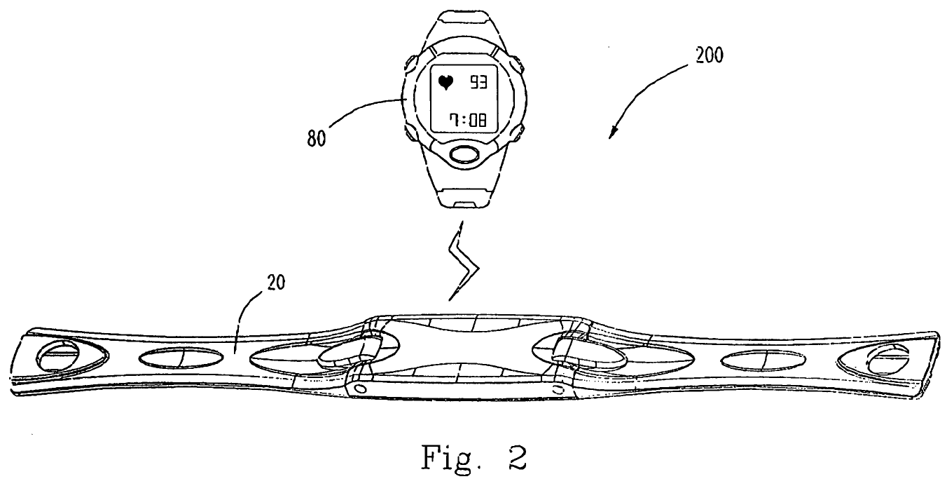

The present invention will now be described more specifically with reference to the following embodiments. Please refer to FIG. 2. FIG. 2 is a diagram illustrating the heart rate monitor device according to a preferred embodiment of the present invention. The heart rate monitor device 200 includes the heart rate monitor 80 and the signal transmitting belt 20. According to a preferred embodiment of the present invention, the heart rate monitor 80 is a wrist watch. The signal transmitting belt 20 transmits a heartbeat signal to the heart rate monitor 80 so as to show a cardiorespiratory information of the user.

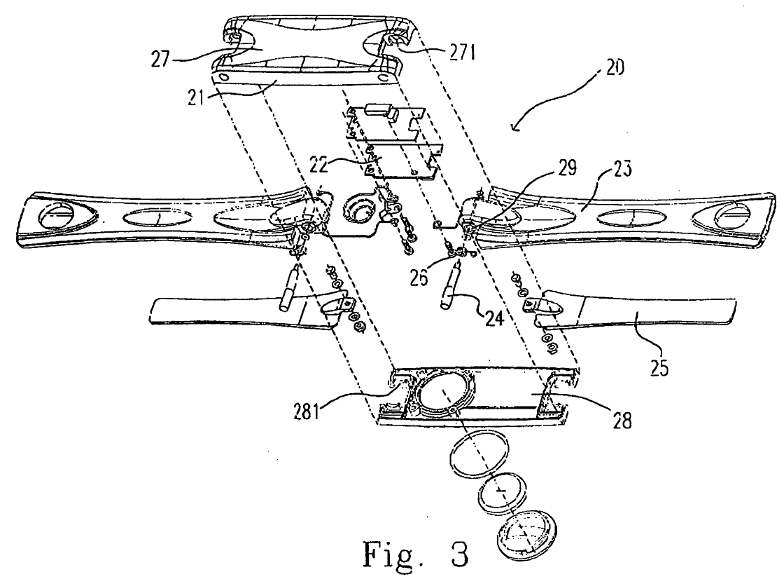

Please refer to FIG. 3. FIG. 3 is a diagram illustrating the structure of the signal transmitting belt according to a preferred embodiment of the present invention. The signal transmitting belt includes the main body 21, the insulating chest belt 23, the connecting element 24, and the conductive chest belt 25. The main body 21 includes the signal transmitter 22 for transmitting the heartbeat signal. The connecting element 24 connects the main body 21 and the insulating chest belt 23, and, it also sustains the pulling tension between the main body 21 and the insulating chest belt 23. The conductive chest belt 25 is mounted on the insulating chest belt 23 for contacting the user's chest (not shown) so as to detect the heartbeat signal and then transmit the heartbeat signal to the signal transmitter 22.

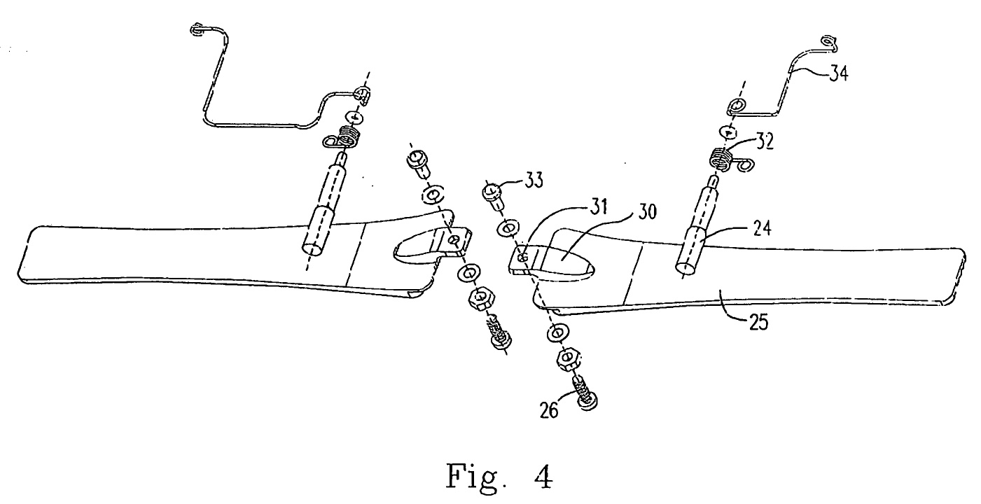

According to a preferred embodiment of the present invention, the connecting element 74 is a round shaft so that the insulating chest belt 23 is rotatable around the main body 21. In addition, the conductive chest belt 25 transmits the heartbeat signal from the user to the signal transmitter 22 through the connecting element 24. Please refer to FIG. 4. FIG. 4 is a diagram illustrating the structure of the connecting shaft and the conductive chest belt according to a preferred embodiment of the present invention. The conductive chest belt 25 further includes the protrudent portion 30 where there is the signal transmitting hole 31 mounted thereon. The heartbeat signal is transmitted through the signal transmitting hole 31. The signal transmitting belt 20 further includes the first spring 32 and the first screw 33. The first spring 32 is mounted on the connecting element 24, while the first screw 33 is screwed onto the signal transmitting hole 31. The first spring 32 is screwed onto the conductive chest belt 25 by the first screw 33 so that the heartbeat signal is transmitted to the connecting element 24. According to a preferred embodiment of the present invention, the signal transmitting belt 20 further includes the second spring 34 and the second screw 26. The second spring 34 is mounted on the connecting element 24, while the second screw 26 is mounted on the main body 21 (FIG. 2). Particularly, the second spring 34 is looped around the connecting element 24 so that the heartbeat signal is transmitted from the connecting element 24 to the signal transmitter 22 of the main body 21.

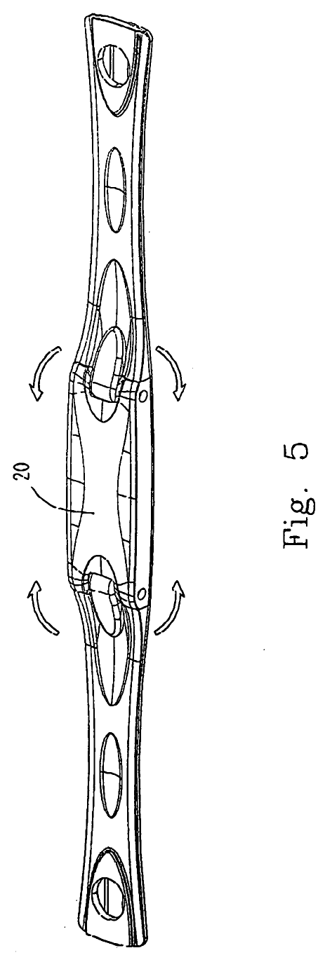

Referring to FIG. 2, the main body 21 further includes the upper cover 27 and the lower cover 28. Also, the upper cover 27 and the lower cover 28 further includes the fixing bearing 271 and the auxiliary bearing 281 respectively so that the connecting element 24 mounted therebetween can be fixed at a certain position. Here the conductive chest belt 25 further includes the axle hole 29 for mounting the connecting element 24 therethrough. The insulating chest belt 23 and the main body 21 are assembled together both through the side portions thereof, which cooperatively forms the axle hole 29 for passing, trough the connecting element 24. Please refer to FIG. 5. FIG. 5 is a diagram showing the outside appearance of the signal transmitting belt according to a preferred embodiment of the present invention. The insulating chest belt 23 has a rotating angle of 180° corresponding to the main body 21, so the signal transmitting belt 20 would have a good flexibility when used. As shown by the arrows in FIG. 5, the insulating chest belt 23 can rotate upwardly and downwardly, both with the rotating angle of 90°. Therefore, an excellent flexibility is provided for the signal transmitting bolt 20, which completely overcomes the drawbacks in the prior art that the signal transmitting belt is not rotatable.

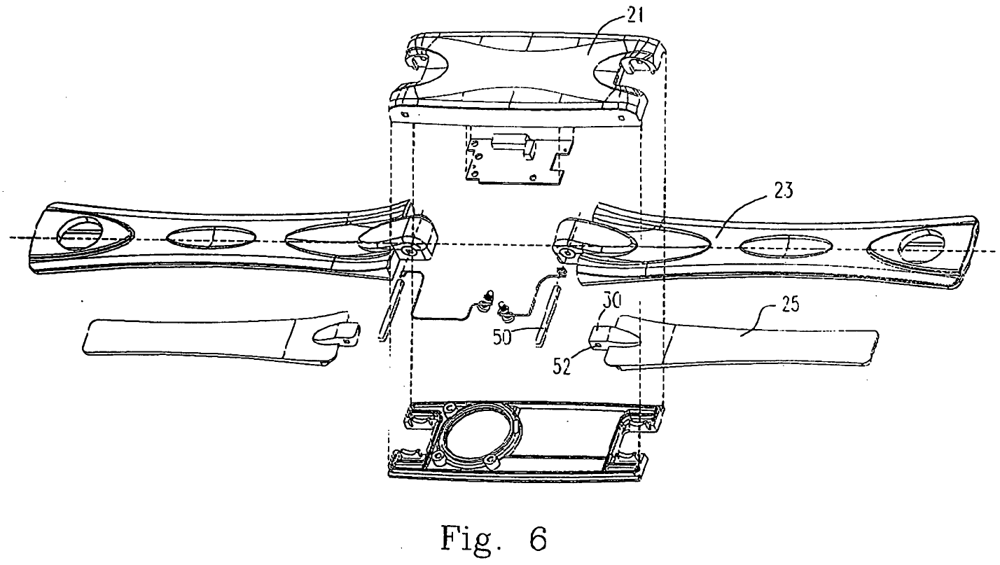

Please refer to FIG. 6. FIG. 6 is a diagram illustrating the structure of the signal transmitting belt according to another preferred embodiment of the present invention. According to another preferred embodiment of the present invention, the structure of the signal transmitting belt can be slightly changed. As shown in FIG. 6, the connecting element 50 is a rectangular shaft. Also, the signal transmitting hole 52 that mounted on the protrudent portion 30 for transmitting the heartbeat signal therethrough is a rectangular hole. The connecting element 50 can directly pass through the signal transmitting hole 52 for connecting the main body 21, the insulating chest belt 23, and the conductive chest belt 25 together. In such an embodiment, the first spring 32 is not needed.

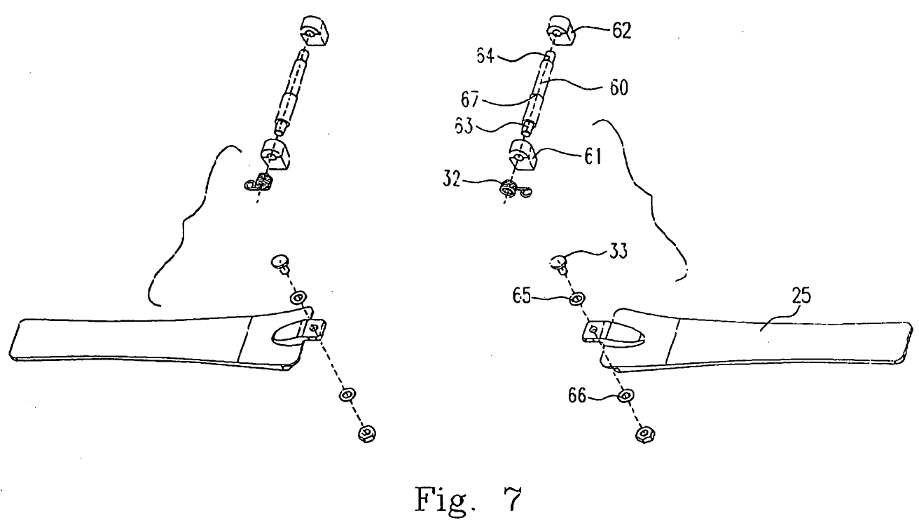

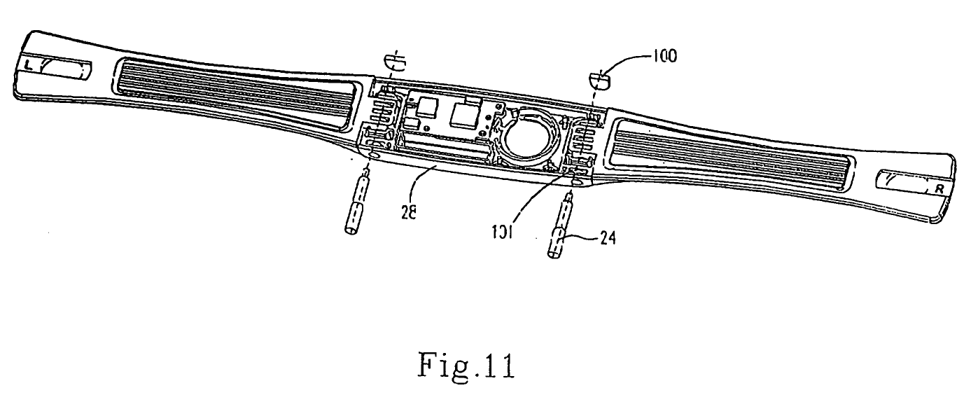

Please refer to FIG. 7. FIG. 7 is a diagram illustrating the structure of the connecting element and the conductive chest belt according to a preferred embodiment of the present invention. The washers 65 and 66 are screwed together with the first spring 32 by the first screw 33. In such a way, the first spring 32 is screwed onto the conductive chest belt 25 under an even pressure. Referring to FIGS. 2 and 7, the first spring 32 is mounted inside the axle hole 29 of the insulating chest belt 23. Furthermore, the connecting element 24 has a flange 67 for being pushed against the first spring 32 mounted inside the axle hole 29. Please refer to FIG. 11. FIG. 11 is a diagram illustrating the structure of the connecting element and the lower cover according to a preferred embodiment of the present invention. The signal transmitting belt 20 further comprises the skidproof slice 100 which is mounted in the indentation 101 of the main body 21 for preventing the connecting element 24 from coming off.

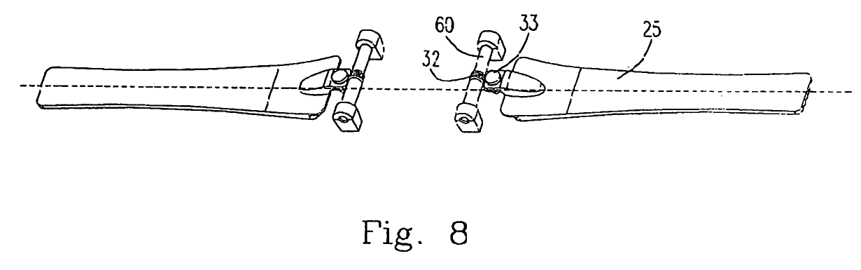

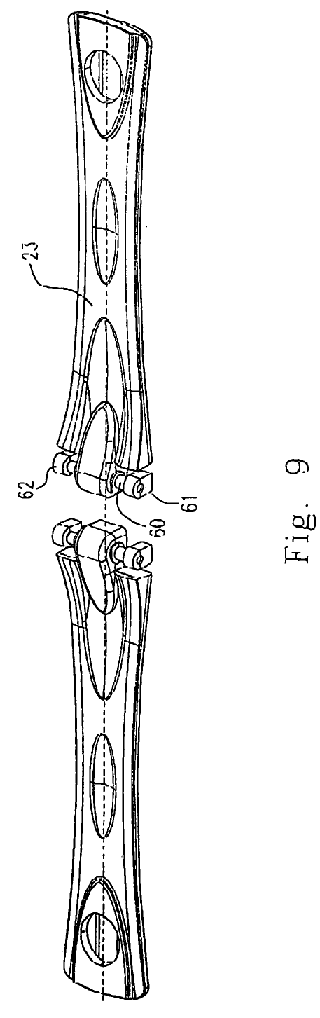

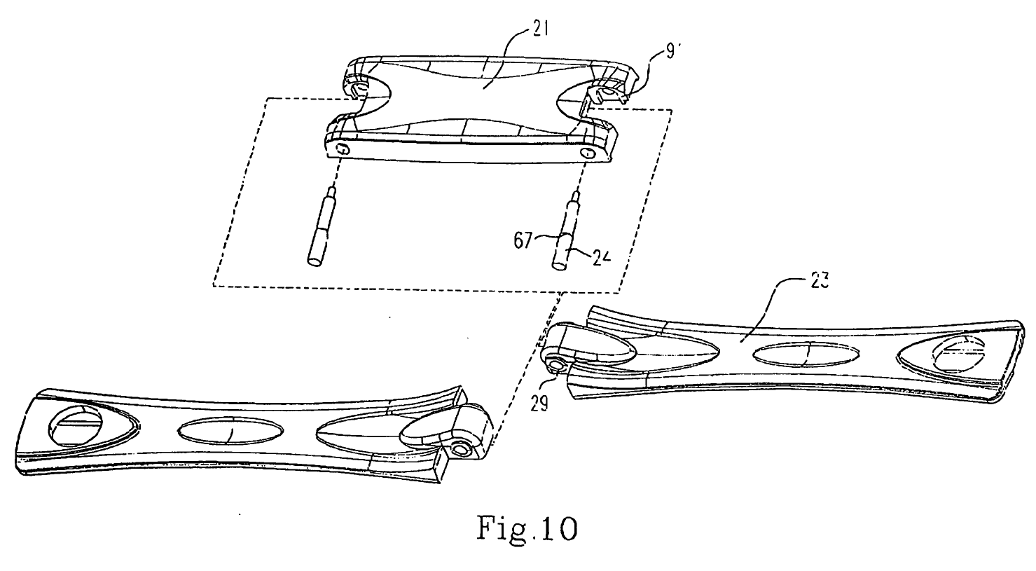

According to another aspect of the present invention, the manufacturing method for a signal transmitting belt of a heart rate monitor is provided. Please refer to FIGS. 7-10. FIGS. 7-10 are diagrams illustrating the manufacturing method for a signal transmitting belt according to a preferred embodiment of the present invention. Generally speaking, the steps are as follows. First, the injection molding assembly (not shown), the conductive chest belt 25, and the first shaft body 60 are provided. Next, the first shaft body 60 and the conductive chest belt 25 are placed inside the injection molding assembly. The molding material is then injected into the injection molding assembly to form the insulating chest bolt 23. Next, the first shaft body 60 is removed and the main body 21 is provided. The second shaft body 24 is then mounted at the same position of the first shaft body 60 for connecting insulating chest belt 23 and the main body 21. Finally, the signal transmitting belt is accomplished.

According to a preferred embodiment of the present invention, the first shaft body 60 further includes two impact bearings 61 and 62, which are mounted at the end of the first shaft body 60. The impact bearings 61 and 62 are used for sustaining the impact force during the injecting. In addition, the first shaft body 60 further includes two junctions 63 and 64 at the end of the first shaft body 60. As shown in FIG. 7, the junctions 63 and 64 are respectively connected with the impact bearings 61 and 62. In the manufacturing process, the impact bearings 61 and 62 are removed together with the first shaft body 60 after the insulating chest belt 23 is formed. In other words, due to the particular design that the first shaft body 60 and the impact bearings 61 and 62 are utilized in the manufacturing process, the accomplished structure of the signal transmitting belt is more accurate and solider.

According to the above, the drawbacks in the conventional signal transmitting belt are not existed in the adaptor provided in the present invention. First, the insulating chest belt has a much wider rotating angle owing to the structure of the connecting element, which allows the belt rotates around the main body of the belt. Therefore, the signal transmitting belt provided in the present invention has a good flexibility so that it is very comfortable for the user. Besides, the structure of the connecting shaft accomplishes a more stable structure since the engagement point between the main body and the insulating chest point will not be easily damaged as described previously. Therefore, the cost for fixing the damage at the engagement point can be saved and the life span of the signal transmitting belt is prolonged. Hence, the present invention not only has a novelty and a progressive nature, but also has an industry utility.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A signal transmitting belt for transmitting a heartbeat signal to a heart rate monitor so as to show a cardiorespiratory information, comprising;
a main body comprising a signal transmitter for transmitting said heartbeat signal;
an insulating chest belt;
a connecting element connecting said main body and said insulating chest belt for sustaining a tension therebetween; and
a conductive chest belt mounted on said insulating chest belt for contacting a chest of a user so as to detect said heartbeat signal and then transmit said heartbeat signal to said signal transmitter.

2. The signal transmitting belt according to claim 1, wherein said connecting element is a connecting shaft.

3. The signal transmitting belt according to claim 1, wherein said connecting element is a round shaft so that said insulating chest belt is rotatable around said main body.

4. The signal transmitting belt according to claim 1, wherein said connecting element is a rectangular shaft.

5. The signal transmitting belt according to claim 1, wherein said conductive chest belt transmits said heartbeat signal from said user to said signal transmitter through said connecting element.

6. The signal transmitting belt according to claim 1, wherein said conductive chest belt further comprises a protrudent portion having a signal transmitting hole thereon for transmitting said heartbeat signal therethrough.

7. The signal transmitting belt according to claim 6, wherein said signal transmitting belt further comprises a first spring mounted on said connecting element and a first screw screwed onto said signal transmitting hole.

8. The signal transmitting belt according to claim 7, wherein said first spring is screwed onto said conductive chest belt by said first screw for transmitting said heartbeat signal to said connecting element.

9. The signal transmitting belt according to claim 8 further comprising a washer screwed together with said first spring by said first screw so that said first spring is strewed onto said conductive chest belt under an even pressure.

10. The signal transmitting belt according to claim 7, wherein said conductive chest belt further comprises an axle hole for mounting said connecting element therethrough.

11. The signal transmitting belt according to claim 10, wherein said first spring is mounted inside said axle hole of said insulating chest belt.

12. The signal transmitting bell according to claim 11, wherein said connecting element has a flange thereon for being pushed against said first spring mounted inside said axle hole.

13. The signal transmitting belt according to claim 10, wherein said insulating chest belt and said main body are assembled together both through side portions thereof to cooperatively form said axle hole for passing through said connecting element.

14. The signal transmitting belt according to clam 1, wherein said signal transmitting belt further comprises a second spring mounted on said connecting element and a second screw mounted on said main body.

15. The signal transmitting belt according to claim 14, wherein said second spring is looped around said connecting element for transmitting said heartbeat signal from said connecting element to Raid signal transmitter of said main body.

16. The signal transmitting belt according to claim 1, wherein said main body further comprises an upper cover and a lower cover.

17. The signal transmitting belt according to claim 16, wherein said upper cover and said lower cover further comprise a fixing hearing and an auxiliary bearing respectively for fixedly mounting said connecting element therebetween.

18. The signal transmitting belt according to claim 1, wherein said signal transmitting belt further comprises a skidproof slice mounted in an indentation of said main body for preventing said connecting element from coming off.

19. The signal transmitting belt according to claim 1, wherein said insulating chest belt has a rotating angle of 180° corresponding to said main body for providing a flexibility to said signal transmitting belt when being used.

20. A signal transmitting belt for transmitting a heartbeat signal to a heart rate monitor so as to show a cardiorespiratory information for a user, comprising:
a main body comprising a signal transmitter therein for transmitting said heartbeat signal;
an insulating chest belt having a conductive sensing element mounted therein for contacting a chest of said user so as to detect said heartbeat signal and then transmit said heartbeat signal to said signal transmitter; and
a connecting element connecting said main body and said insulating chest belt for sustaining a tension therebetween.
